SEQUENCE LISTING (5 sequences)

SEQ ID NO: 1 - protein, 308 aa, Salipaludibacillus agaradhaerens
AGFYVDGNTLYDANGQPFVMRGINHGHAWYKDTASTAIPAIAEQGANTIRIVLSDGGQWEKDDIDTIREVIELAEQNKMVAVVEVHDATGRDSRSDLNRAVDYWIEMKDALIGKEDTVIINIANEWYGSWDGSAWADGYIDVIPKLRDAGLTHTLMVDAAGWGQYPQSIHDYGQDVFNADPLKNTMFSIHMYEYAGGDANTVRSNIDRVIDQDLALVIGEFGHRHTDGDVDEDTILSYSEETGTGWLAWSWKGNSTEWDYLDLSEDWAGQHLTDWGNRIVHGADGLQETSKPSTVFTDDNGGHPEPPT

SEQ ID NO: 2 - protein, 296 aa, Paenibacillus polymyxa
ASGFYVSGTNLYDSTGKPFVMRGVNHAHTWYKNDLYTAIPAIAKTGANTVRIVLSNGNQYTKDDINSVKNIISLVSNHKMIAVLEVHDATGKDDYASLDAAVNYWISIKDALIGKEDRVIVNIANEWYGSWNGGGWADGYKQAIPKLRNAGIKNTLIVDCAGWGQYPQSINDFGKSVFAADSLKNTVFSIHMYEFAGKDVQTVRTNIDNVLYQGLPLIIGEFGGYHQGADVDETEIMRYGQSKSVGWLAWSWYGNSSNLNYLDLVTGPNGNLTDWGRTVVEGANGIKETSKKAGIF

SEQ ID NO: 3 - protein, 299 aa, Paenibacillus lentus
ASGFYVSGTILCDSTGNPFKIRGINHAHSWFKNDSATAMEAIAATGANTVRIVLSNGQQYAKDDANTVSNLLSLANQHKLIAILEVHDATGSDSVSALDHAVDYWIEMKNVLVGKEDRVLINIANEWYGTWDSNGWADGYKSAIPKLRNAGINHTLIVDAAGWGQYPQSIVDKGNEVFNSDPLRNTIFSIHMYEYAGGNADMVRANIDQVLNKGLAVIIGEFGHYHTGGDVDETAIMSYTQQKGVGWLAWSWKGNGAEWLYLDLSYDWAGNHLTEWGETIVNGANGLKATSTRAPIFGN

SEQ ID NO: 4 - protein, 307 aa, Xanthomonas campestris pv. Campestris
GLSVSGTQLKESNGNTLILRGINLPHAWFADRTDAALAQIAATGANSVRVVLSSGHRWNRTPEAEVARIIARCKALGLIAVLEVHDTTGYGEDGAAGSLANAASYWTSVRTALVGQEDYVIINIGNEPFGNQLSASEWVNGHANAIATLRGAGLTHALMVDAPNWGQDWQFYMRDNAAALLARDSRRNLIFSVHMYEVFGSDAVVDSYLRTFRSNNLALVVGEFGADHRGAPVDEAAIMRRAREYGVGYLGWSWSGNDSSTQSLDIVLGWDPARLSSWGRSLIQGPDGIAATSRRARVFGARVRAME

SEQ ID NO: 5 - protein, 300 aa, Ruminiclostridium sp
GGFKVEGTKLLDANGKEFIMRGINHAHTWYLDEDTTAIKAIAETGSNVVRVVCSDGEQWTKDTEDMLETVIDLCIDNEMIAVVEVHDATGKDDKTALDKATDYWIEMKNALIGKEQYVILNIANEWTGGWNGELWRDGYTESIPKLREAGIKNTILVDAAGWGQYAKSIGDYGKEVFDSDPDKNTMFAVHMYGTAGKNSSVIEKNLKYATDNGLCVIVGEFGYTHTDGDVDEAFIMKYCQDNGIGYIGWSWKGNSGGVEYLDIANSWDGSVLSADWGENLVNGENGIKQTSVKCSVFTKE

(19) United States
(12) Patent Application Publication
(10) Pub. No.: US 2026/0078204 A1
(43) Pub. Date: Mar. 19, 2026

(54) COMPOSITION CONTAINING PARTIALLY HYDROLYZED GUAR GUM AND METHOD FOR PREPARING THE SAME

(71) Applicant: ZYMEBASE INC., Shanghai (CN)

(72) Inventors: Cong TAN, Shanghai (CN); Kun ZHONG, Shanghai (CN); Yuan CHEN, Shanghai (CN); Han ZHANG, Shanghai (CN)

(73) Assignee: ZYMEBASE INC., Shanghai (CN)

(21) Appl. No.: 19/379,797

(22) Filed: Nov. 5, 2025

Related U.S. Application Data
(63) Continuation of application No. PCT/CN2023/095287, filed on May 19, 2023.

(51) Int. Cl. C08B 37/00; C12P 19/04; C12P 19/14

(52) U.S. Cl. CPC C08B 37/0096; C12P 19/04; C12P 19/14

(57) ABSTRACT

The present disclosure disclosed a method for preparing a PHGG sample and a composition containing the PHGG sample. The method may include performing enzymatic hydrolysis on a guar gum sample using a β-mannanase to obtain a reaction mixture. The method may also include processing the reaction mixture to obtain the PHGG sample. The PHGG sample may include a plurality of polysaccharides, and more than 70% by weight of the plurality of polysaccharides may have molecular weights higher than 5,000 Daltons. The PHGG sample includes one or more oligosaccharides that have degrees of polymerization (DP) less than or equal to 10, and in the PHGG sample, a mass concentration of the one or more oligosaccharides having DP less than or equal to 10 is less than 3%.

### CROSS-REFERENCE TO RELATED APPLICATIONS

This application is a continuation of International Patent Application No. PCT/CN2023/095287, filed on May 19, 2023, the entire contents of which are incorporated herein by reference.

### SEQUENCE LISTING

The instant application contains a Sequence Listing which has been submitted electronically in XML format and is hereby incorporated by reference in its entirety. The XML file, created on May 19, 2023, is named “20770-0003WO00”, and is 6,535 bytes in size.

### TECHNICAL FIELD

The present disclosure relates to the field of bioengineering technology, and specifically relates to a composition including a partially hydrolyzed guar gum (PHGG) sample and a method for preparing the PHGG.

### BACKGROUND

Guar gum (also referred to as a guar gum sample) is a galactomannan extracted from seeds of an annual leguminous plant. Generally, a guar gum sample includes guar gum molecules, which are high molecular polysaccharides with molecular weights ranging from hundreds of thousands to millions of Daltons. Guar gum has multifarious applications such as a food additive and dietary fiber. For example, as a water-soluble dietary fiber, guar gum has physiological functions such as prebiotics. However, guar gum also has limitations for its use in food products due to high viscosity even at a relatively low concentration and the property of tending to form a gel. For example, a viscosity of an aqueous solution of a guar gum sample under 1% by weight can be as high as 2000-3000 mPa·s.

In partially hydrolyzed guar gum (PHGG) (also referred to as a PHGG sample), the average molecular weight of guar gum is reduced, as compared to regular guar gum, resulting in changes in the viscosity, the solubility, or other physicochemical properties of the guar gum sample. PHGG also has multiple bioactivities that regular guar gum does not have. Thus, PHGG has been used in cereals, juices, shakes, yogurt, meal replacements, soups, and baked goods and as a fiber source in enteral nutrition products.

However, though oligosaccharides in PHGG have low viscosity and good solubility, the oligosaccharides do not have bioactivities (e.g., a biological regulatory activity) that the regular guar gum has. Therefore, it is desirable to provide an effective method for preparing a PHGG sample, which can balance the viscosity and the bioregulatory activity of PHGG for better use of PHGG.

The information disclosed in this background is only for the enhancement of understanding of the general background of the present disclosure and should not be taken as an acknowledgment or any form of suggestion that this information forms the prior art already known to a person skilled in the art.

### SUMMARY

In one aspect of the present disclosure, a method for preparing a partially hydrolyzed guar gum (PHGG) sample is provided. The method may include performing enzymatic hydrolysis on a guar gum sample using a β-mannanase to obtain a reaction mixture. The method may also include processing the reaction mixture to obtain the PHGG sample. The PHGG sample may include a plurality of polysaccharides, and more than 70% by weight of the plurality of polysaccharides may have molecular weights higher than 5,000 Daltons.

In some embodiments, wherein the β-mannanase may include a glycosyl hydrolase 5 family β-mannanase.

In some embodiments, the β-mannanase may include a glycosyl hydrolase 5 family β-mannanase absent of carbohydrate-binding modules (CBMs).

In some embodiments, the β-mannanase may include at least one of SagMan derived from Salipaludibacillus agaradhaerens, PpoMan derived from Paenibacillus polymyxa, PleMan derived from Paenibacillus lentus, XccMan derived from Xanthomonas campestris pv. campestris, or RspMan derived from Ruminiclostridium sp. The SagMan may have an amino acid sequence of SEQ ID NO: 1. PpoMan may have an amino acid sequence of SEQ ID NO: 2. PleMan may have an amino acid sequence of SEQ ID NO: 3. XccMan may have an amino acid sequence of SEQ ID NO: 4. RspMan may have an amino acid sequence of SEQ ID NO: 5

In some embodiments, wherein more than 80% by weight of the plurality of polysaccharides may have molecular weights higher than 5,000 Daltons.

In some embodiments. More than 85% by weight of the plurality of polysaccharides may have molecular weights higher than 5,000 Daltons.

In some embodiments, the PHGG sample may include one or more oligosaccharides that have degrees of polymerization (DP) less than or equal to 10, and a mass concentration of all the one or more oligosaccharides in the PHGG sample may be less than 3%.

In some embodiments, the one or more one or more oligosaccharides may have molecular weights less than 1,800 Daltons.

In some embodiments, the performing the enzymatic hydrolysis on the guar gum sample using the β-mannanase to obtain the reaction mixture may include preparing a mannanase mixture including the β-mannanase and having a preset PH; adding, by stirring the mannanase mixture under a preset temperature, the guar gum sample into the mannanase mixture to obtain a gel mixture, the guar gum sample being in powder; and reacting the gel mixture for a preset time to obtain the reaction mixture.

In some embodiments, a mass concentration of the guar gum sample in the gel mixture may be from 5% to 20%.

In some embodiments, the mass concentration of the guar gum sample in the gel mixture may be from 8% to 20%.

In some embodiments, the mass concentration of the guar gum sample in the gel mixture may be from 10% to 20%.

In some embodiments, the preset time may be from 1 hour to 24 hours.

In some embodiments, the preset time may be from 1 hour to 4 hours.

In some embodiments, the PHGG sample may have an average molecular weight from 10 kDa to 30 kDa.

In some embodiments, the PHGG sample may have the average molecular weight from 15 kDa to 25 kDa.

In some embodiments, the processing the reaction mixture may include inactivating the β-mannanase in the reaction mixture.

In some embodiments, the processing the reaction mixture may further include performing at least one of a centrifugal operation, a filtering operation, a sterilization operation, a concentration operation, or a dry operation on the reaction mixture.

In some embodiments, the method may further include analyzing the PHGG sample by at least one of size exclusion chromatography (SEC), high performance liquid chromatography (HPLC), a reducing sugar analyzation, or ion chromatography (IC).

In another aspect of the present disclosure, a composition containing a partially hydrolyzed guar gum (PHGG) sample is provided. The PHGG sample may be prepared by performing enzymatic hydrolysis on a guar gum sample using a β-mannanase to obtain a reaction mixture and processing the reaction mixture. The PHGG sample may include a plurality of polysaccharides, and more than 70% by weight of the plurality of polysaccharides may have molecular weights higher than 5,000 Daltons.

In some embodiments, the β-mannanase may include a glycosyl hydrolase 5 family β-mannanase.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. It should be noted that the drawings are not to scale. These embodiments are non-limiting exemplary embodiments, in which like reference numerals represent similar structures throughout the several views of the drawings, and wherein:

FIG. 1 is a flowchart illustrating an exemplary process for preparing a PHGG sample according to some embodiments of the present disclosure;

FIGS. 2A-2E are exemplary graphs showing exemplary molecular weight analyses of PHGG samples according to some embodiments of the present disclosure;

FIGS. 3A-3E are exemplary graphs showing exemplary oligosaccharide analyses of PHGG samples according to some embodiments of the present disclosure;

FIG. 4 is an exemplary graph showing an exemplary SEC analysis of PHGG samples according to some embodiments of the present disclosure;

FIG. 5 is an exemplary graph showing exemplary average molecular weights of PHGG samples according to some embodiments of the present disclosure; and

FIG. 6 is an exemplary graph showing an exemplary PAHBAH reducing sugar assay of PHGG samples according to some embodiments of the present disclosure.

### DETAILED DESCRIPTION

The following description is presented to enable any person skilled in the art to make and use the present disclosure and is provided in the context of a particular application and its requirements. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but is to be accorded the widest scope consistent with the claims.

The terminology used herein is to describe particular example embodiments only and is not intended to be limiting. As used herein, the singular forms “a,” “an,” and “the” may be intended to include the plural forms as well, unless the context clearly indicates otherwise. It will be further understood that the terms “comprises,” “comprising,” “includes,” “including,” “consist of,” and/or “consist essentially of” when used in this specification, specify the presence of stated features, integers, steps, operations, elements, compositions, and/or components, but do not preclude the presence or addition of one or more other features, integers, steps, operations, elements, compositions, components, and/or groups thereof. For example, a method of the present disclosure may comprise or include essential operations as well as optional or additional operations described herein, but only if the optional or additional operations do not materially alter the basic and novel characteristics of the claimed product of the method.

As used herein, the number of significant digits conveys neither a limitation on the indicated amounts nor the accuracy of the measurements. All numerical amounts should be understood to be modified by the word “about” unless otherwise specifically indicated. As used herein, the term “about” and its grammatical equivalents in relation to a reference numerical value and its grammatical equivalents as used herein can include a range of values plus or minus 10% from that value, such as a range of values plus or minus 10%, 9%, 8%, 7%, 6%, 5%, 4%, 3%, 2%, or 1% from that value. For example, the amount of 10 may include amounts from 9 to 11. All numeric ranges are inclusive of narrower ranges and combinable; delineated upper and lower range limits are interchangeable to create further ranges not explicitly delineated.

The term “composition containing a PHGG sample” may refer to any composition that contains the PHGG sample. The compositions containing the PHGG sample herein may also include a variety of optional ingredients that are known for use, as long as the optional ingredient(s) do not unduly alter product stability, aesthetics, or performance of the PHGG sample. The term “concentration” may refer to a mass concentration, unless otherwise specifically indicated.

Unless otherwise defined, all technical and scientific terms used in the present disclosure have the same meaning as commonly understood by one of ordinary skill in the art to which the present disclosure relates. Although only the preferred methods and materials are described herein, any methods and materials similar or equivalent to those described in the present disclosure can also be used in the practice or testing of the present disclosure. All documents mentioned in the present disclosure are incorporated by reference for the purpose of disclosing and describing the methods and/or materials in connection with which the documents are referred. In the event of a conflict with any incorporated document, the content of the present disclosure controls.

According to some embodiments of the present disclosure, a method for preparing a PHGG sample is provided. The method may include performing enzymatic hydrolysis on a guar gum sample using a β-mannanase to obtain a reaction mixture. The method may also include processing the reaction mixture to obtain the PHGG sample. The obtained PHGG sample may include a plurality of polysaccharides, and more than 70% by weight of the plurality of polysaccharides may have molecular weights higher than 5,000 Daltons (i.e., Da). According to some embodiments of the present disclosure, a composition containing the PHGG sample is provided.

Generally, a PHGG sample with an average molecular weight in a specified range of [5,000 Daltons, 40,000 Daltons] may have better physicochemical properties and better bioactivities than a PHGG sample with an average molecular weight outside the specified range. However, oligosaccharides in the PHGG sample may affect the balance of the physicochemical property and the bioactivity of the PHGG sample. For example, the fewer the oligosaccharides in the PHGG sample, the better the balance between the physicochemical property and the bioactivity of the PHGG sample. According to the method disclosed in the present disclosure, the average molecular weight of the PHGG sample may be controlled to be in the specific range, which improves the property of the PHGG sample. In addition, according to the method disclosed in the present disclosure, oligosaccharides in the PHGG sample may have a low weight ratio, which further improves the property of the PHGG sample. With the improved property of the PHGG sample, the PHGG sample can be ingested without food palatability degradation even when added in large amounts and retains the high biological regulatory activity of the guar gum sample.

The method may include performing enzymatic hydrolysis on a guar gum sample using a β-mannanase to obtain a reaction mixture. The method may also include processing the reaction mixture to obtain the PHGG sample. The obtained PHGG sample may include a plurality of polysaccharides, and more than 70% by weight of the plurality of polysaccharides may have molecular weights higher than 5,000 Daltons.

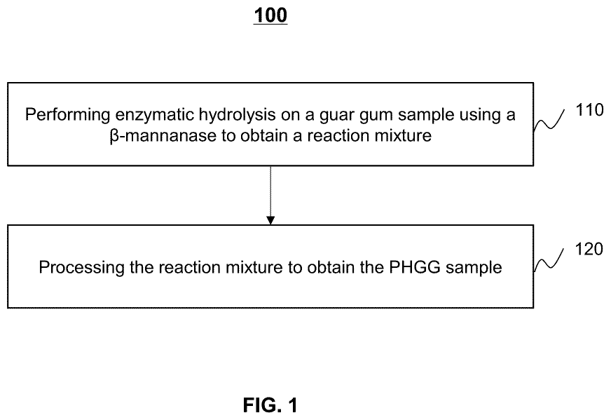

FIG. 1 is a flowchart illustrating an exemplary process for preparing a PHGG sample. As shown in FIG. 1, the process 100 may include operations 110 and 120. It should be noted that the operations of the illustrated process presented below are intended to be illustrative. In some embodiments, the process 100 may be accomplished with one or more additional operations not described.

In 110, enzymatic hydrolysis may be performed on a guar gum sample using a β-mannanase to obtain a reaction mixture.

Hemicellulose is the second most abundant polymer found in nature, which is usually present together with lignin and cellulose in the plant cell walls. It is estimated that hemicellulose is a third of the total components in plants, for example, hemicellulose makes up 25-30% of the total weight of dry wood. Hetero-1,4-β-D-mannans and hetero-1,4-β-D-xylans are two most significant types of hemicelluloses.

In grasses and hardwoods, xylan is the major hemicellulose component, while in softwoods, plant fruits and seeds the hemicellulose is mainly present in the form of mannan. Mannan mainly appears in four different forms: linear mannan, galactomannan, glucomannan, and galactoglucomannan. Due to heterogenous nature of mannan, the biodegradation of mannan may require a close association and synergy among β-mannanase (EC 3.2.1.78), β-mannosidase (EC 3.2.1.25), acetyl mannan esterase (EC 3.1.1.6), β-glucosidase (EC 3.2.1.21), and α-galactosidase (EC 3.2.1.22) to break the main and the side chains of mannan. β-mannanase is a major mannan degrading enzyme.

In some embodiments, the β-mannanase may be isolated/derived from plants, animals, microbes, or the like, or any combination thereof. Most of commercial β-mannanases may be produced from the microbes due to the high stability, high production within limited time and space, cost effectiveness, and ease of genetic manipulation. The microbes may include bacteria, fungi, actinomycetes, etc. For example, the β-mannanase may be an enzyme derived from bacteria such as Salipaludibacillus agaradhaerens, Bacillus species, Klebsiella Oxytoca, Thhermotoga neapolitana, Thermoanaerobacterium polysaccharolytthcum, Clostridiu, cellulovorans, Acinetobacter sp., or Cellulomonas fimi. As another example, the β-mannanase may be an enzyme derived from fungi such as Aspergillus, Rhizopus, Trichoderma sp., Penicillium sp., Penicillium purpurogenum, Axhaetomium sp., Chaetomium sp., Humicola insolens, Thielavia arenaria, or Rhizomucor miehei. As still another example, the β-mannanase may be an enzyme derived from actinomycetes such as Streptomyces sp. or Nocardiopsis sp. In some embodiments, not being subject to any limitation, the process to produce the β-mannanase may include any possible method. For instance, the β-mannanase may be produced by using merely by way of example, a gene encoding the β-mannanase from a bacterium may be synthesized. An enzyme of the β-mannanase may be produced in Bacillus subtilis cells using an induction system disclosed in: “A wheat bran inducible expression system for the efficient production of α-L-arabinofuranosidase in Bacillus subtilis” (Ji M et al., Enzyme Microb Technol. 2021 March; 144:109726), which is being incorporated by reference.

In some embodiments, the β-mannanase used herein may include a glycosyl hydrolase (GH) 5 (cellulase A) family β-mannanase. For example, the β-mannanase may include a GH 5 family β-mannanase that is absent of carbohydrate-binding modules (CBMs). For instance, the β-mannanase may include or maybe at least one of the SagMan enzyme (“SagMan”), the PpoMan enzyme (“PpoMan”), the PleMan enzyme (“PleMan”), the XccMan enzyme (“XccMan”), or the RspMan enzyme (“RspMn”). As another example, the β-mannanase may include or maybe any other enzyme whose sequence and a sequence of at least one of the SagMan enzyme, the PpoMan enzyme, the PleMan enzyme, the XccMan enzyme, or the RspMan enzyme have a homology greater than or equal to 90%.

The SagMan may be derived from, e.g., Salipaludibacillus agaradhaerens (Uniprot #: G1K3N4, GH5 mannanase from Salipaludibacillus agaradhaerens). The SagMan may include a polypeptide having an amino acid sequence of SEQ ID No. 1, which is as follows:

AGFYVDGNTLYDANGQPFVMRGINHGHAWYKDTASTAIPAIAEQGANTI
RIVLSDGGQWEKDDIDTIREVIELAEQNKMVAVVEVHDATGRDSRSDLN
RAVDYWIEMKDALIGKEDTVIINIANEWYGSWDGSAWADGYIDVIPKLR
DAGLTHTLMVDAAGWGQYPQSIHDYGQDVFNADPLKNTMFSIHMYEYAG
GDANTVRSNIDRVIDQDLALVIGEFGHRHTDGDVDEDTILSYSEETGTG
WLAWSWKGNSTEWDYLDLSEDWAGQHLTDWGNRIVHGADGLQETSKPST
VFTDDNGGHPEPPT.

The PpoMan may be derived from, e.g., Paenibacillus polymyxa (Uniprot #: E3EGK4, GH5 mannanase from Paenibacillus polymyxa). The PpoMan may include a polypeptide having an amino acid sequence of SEQ ID No. 2, which is as follows:

ASGFYVSGTNLYDSTGKPFVMRGVNHAHTWYKNDLYTAIPAIAKTGANT
VRIVLSNGNQYTKDDINSVKNIISLVSNHKMIAVLEVHDATGKDDYASL
DAAVNYWISIKDALIGKEDRVIVNIANEWYGSWNGGGWADGYKQAIPKL
RNAGIKNTLIVDCAGWGQYPQSINDFGKSVFAADSLKNTVFSIHMYEFA
GKDVQTVRTNIDNVLYQGLPLIIGEFGGYHQGADVDETEIMRYGQSKSV
GWLAWSWYGNSSNLNYLDLVTGPNGNLTDWGRTWVEGANGIKETSKKAG
IF.

The PleMan may be derived from, e.g., Paenibacillus lentus (Uniprot #: A0A3S8RW94, GH5 mannanase from Paenibacillus lentus). The PleMan may include a polypeptide having an amino acid sequence of SEQ ID No. 3, which is as follows:

ASGFYVSGTILCDSTGNPFKIRGINHAHSWFKNDSATAMEAIAATGANT
VRIVLSNGQQYAKDDANTVSNLLSLANQHKLIAILEVHDATGSDSVSAL
DHAVDYWIEMKNVLVGKEDRVLINIANEWYGTWDSNGWADGYKSAIPKL
RNAGINHTLIVDAAGWGQYPQSIVDKGNEVFNSDPLRNTIFSIHMYEYA
GGNADMVRANIDQVLNKGLAVIIGEFGHYHTGGDVDETAIMSYTQQKGV
GWLAWSWKGNGAEWLYLDLSYDWAGNHLTEWGETIVNGANGLKATSTRA
PIFGN.

The XccMan may be derived from, e.g., Xanthomonas campestris pv. Campestris (Uniprot #: Q8P9S0, GH5 mannanase from Xanthomonas campestris pv. campestris). The XccMan may have a polypeptide having an amino acid sequence of SEQ ID No. 4, which is as follows:

GLSVSGTQLKESNGNTLILRGINLPHAWFADRTDAALAQIAATGANSVR
VVLSSGHRWNRTPEAEVARIIARCKALGLIAVLEVHDTTGYGEDGAAGS
LANAASYWTSVRTALVGQEDYVIINIGNEPFGNQLSASEWVNGHANAIA
TLRGAGLTHALMVDAPNWGQDWQFYMRDNAAALLARDSRRNLIFSVHMY
EVFGSDAVVDSYLRTFRSNNLALVVGEFGADHRGAPVDEAAIMRRAREY
GVGYLGWSWSGNDSSTQSLDIVLGWDPARLSSWGRSLIQGPDGIAATSR
RARVFGARVRAME.

The RspMan may be derived from, e.g, Ruminiclostridium sp (GenBank: MBS1418002.1, GH5 mannanase from Ruminiclostridium sp.). The RspMan may have a polypeptide having an amino acid sequence of SEQ ID NO: 5, which is as follows:

GGFKVEGTKLLDANGKEFIMRGINHAHTWYLDEDTTAIKAIAETGSNVV
RVVCSDGEQWTKDTEDMLETVIDLCIDNEMIAVVEVHDATGKDDKTALD
KATDYWIEMKNALIGKEQYVILNIANEWTGGWNGELWRDGYTESIPKLR
EAGIKNTILVDAAGWGQYAKSIGDYGKEVFDSDPDKNTMFAVHMYGTAG
KNSSVIEKNLKYATDNGLCVIVGEFGYTHTDGDVDEAFIMKYCQDNGIG
YIGWSWKGNSGGVEYLDIANSWDGSVLSADWGENLVNGENGIKQTSVKC
SVFTKE.

As used herein, the enzymatic hydrolysis may refer to a limited hydrolytic process of hydrolyzing a polysaccharide (e.g., the guar gum sample) using an enzyme (e.g., the β-mannanase). As used herein, the guar gum sample may refer to a linear polysaccharide having chains of β-1,4-linked D-mannose backbone and α-1,6-linked D-galactose side chains. In some embodiments, the guar gum sample may be in powder. The β-mannanase may also be referred to as a β-1,4-D-mannanase and be capable of hydrolyzing β-1,4-linkages in guar gum under conditions of wide range of pH (e.g., 5-8) and temperature (e.g., 40° C.-70° C.).

In some embodiments, the performing the enzymatic hydrolysis on the guar gum sample using the β-mannanase to obtain the reaction mixture may include a plurality of operations/steps. For example, firstly, a mannanase mixture (e.g., a culture medium) including the β-mannanase may be prepared. The β-mannanase in the mannanase mixture may be at a preset concentration. The preset concentration may range from 0.0001%-0.2%. The mannanase mixture may be at a preset PH. The preset PH may range from 5 to 8. Secondly, the guar gum sample may be added, by stirring the mannanase mixture under a preset temperature, into the mannanase mixture to obtain a gel mixture. The preset temperature may range from 40° C. to 70° C. Thirdly, the gel mixture may be reacted for a preset time to obtain the reaction mixture. The preset time may range from 1 hour to 24 hours (e.g., range from 1 hour to 4 hours). The reaction mixture may include limited/partially hydrolyzed guar gum and unreacted components (e.g., the β-mannanase, unreacted guar gum, etc.). The viscosity of the reaction mixture may range from 20 mPa·s to 120 mPa·s.

According to adding the guar gum sample in powder to the mannanase mixture by stirring, a mass concentration of the guar gum sample in the gel mixture may reach an improved concentration. The improved concentration may range from 5% to 20%. However, generally, a gel mixture prepared without stirring, e.g., by directly inputting the guar gum sample into the mannanase mixture, may correspond to a limited concentration of the guar gum sample that is less than the improved concentration, as the guar gum sample under a concentration of 0.5%-1% may form a gel. For example, the mass concentration of the guar gum sample in the gel mixture may be from 8% to 20%. As another example, the mass concentration of the guar gum sample in the gel mixture may be from 10% to 20%. In addition, since a catalytic efficiency of the β-mannanase (e.g., the SagMan) may be negative to molecular weight of the reactant (e.g., the guar gum sample), as the enzymatic hydrolysis proceeds, the guar gum sample may be further hydrolyzed and the hydrolyzed guar gum may have smaller molecular weight, thereby the reaction rate of the enzymatic hydrolysis may be reduced. Accordingly, the enzymatic hydrolysis of the guar gum sample may be avoided to occur overreaction to generate more oligosaccharides with small molecular weights. However, in current enzymatic hydrolysis process, as the enzymatic hydrolysis proceeds, the concentration of the oligosaccharides may be increased rapidly, which is hard to be controlled and also results in subsequent complex process for removing the oligosaccharides. For example, by using the SagMan disclosed herein, even though the enzymatic hydrolysis proceeds for 24 h, the mass concentration of all the oligosaccharides in the PHGG sample may be still less than 3%, while in current enzymatic hydrolysis process, when the enzymatic hydrolysis proceeds for 24 h, the mass concentration of all the oligosaccharides in the PHGG sample may reach about 25%.

In 120, the reaction mixture may be processed to obtain the PHGG sample.

In some embodiments, at least one of an inactivation operation, a centrifugal operation, a filtering operation, a sterilization operation, a concentration operation, or a dry operation may be performed on the reaction mixture. For example, the inactivation operation may be performed on the reaction mixture to inactivate the β-mannanase in the reaction mixture. As another example, the centrifugal operation and the filtering operation may be performed on the reaction mixture to remove a portion of the unreacted components (e.g., the unreacted guar gum, insoluble substances, etc.) in the reaction mixture. As still another example, the sterilization operation may be performed using an ultra-high temperature instantaneous sterilization (UIT) technique, e.g., after the inactivation operation, the centrifugal operation and the filtering operation. As further another example, the dry operation may be performed to obtain the PHGG sample in powder. As a further example, the concentration operation may be performed before the dry operation to obtain concentrated sample for drying.

In some embodiments, the obtained PHGG sample may have an average molecular weight from 10 kDa to 30 kDa. For example, the PHGG sample may have an average molecular weight from 15 kDa to 25 kDa. As another example, the PHGG sample may have an average molecular weight from 17 kDa to 20 kDa. In some embodiments, the obtained PHGG sample may include a plurality of polysaccharides and one or more oligosaccharides. Each of the plurality of polysaccharides may have a degree of polymerization (DP) greater than 10. More than 70% by weight of the plurality of polysaccharides may have molecular weights higher than 5,000 Daltons. For example, more than 80% by weight of the plurality of polysaccharides may have molecular weights higher than 5,000 Daltons. As another example, more than 85% by weight of the plurality of polysaccharides may have molecular weights higher than 5,000 Daltons. Each of the one or more oligosaccharides may have a degree of polymerization (DP) less than or equal to 10. The one or more oligosaccharides may have molecular weights less than 1,800 Daltons. A mass concentration of all the one or more oligosaccharides in the PHGG sample may be less than 3%. For example, the mass concentration of all the one or more oligosaccharides in the PHGG sample may be less than 2%. As still another example, the mass concentration of all the one or more oligosaccharides in the PHGG sample may be less than 1%. In some embodiments, an oligosaccharide having a DP equal to 1 or 2 may also be referred to as a monosaccharide or a disaccharide. Most monosaccharides, disaccharides and oligosaccharides may belong to reducing sugars. The obtained PHGG sample may do not include any detectable amount of reducing sugars. Alternatively, a mass concentration of reducing sugars in the obtained PHGG sample may be less than or equal to a preset threshold (e.g., 0.4%, 0.3%, 0.2%, 0.05%, etc.).

In some embodiments, the PHGG sample may be analyzed by at least one of size exclusion chromatography (SEC), high performance liquid chromatography (HPLC), or a reducing sugar analyzation, or ion chromatography (IC). For example, the PHGG sample may be analyzed by the SEC to determine a molecular weight (e.g., the average molecular weight) of the PHGG sample. As another example, the PHGG sample may be analyzed by the HPLC and/or the reducing sugar analyzation to determine a concentration of the one or more oligosaccharides in the PHGG sample. As still another example, the PHGG sample may be analyzed by the IC to determine the types of the plurality of polysaccharides and/or the one or more oligosaccharides in the PHGG sample. More descriptions regarding the production and/or analysis of the PHGG sample may be found elsewhere in the present disclosure, e.g., the following examples.

In some embodiments, the guar gum may be used as a thickener or emulsifier. However, the guar gum may be limited to be used only in small quantities in different food products, as in large quantities, high-viscosity guar gum can cause obstruction of the esophagus or small bowel due to its ability to expand to 10-20 times its size. According to some embodiments of the present disclosure, the obtained PHGG sample may have improved properties than the regular guar gum sample (e.g., the guar gum sample before hydrolysis). In some embodiments, the PHGG sample may have an improved molecular weight in comparison of the guar gum sample. For instance, more than 70% by weight of the plurality of polysaccharides in the PHGG sample may have molecular weights higher than 5,000 Daltons and a mass concentration of all the one or more oligosaccharides in the PHGG sample may be less than 3%, while the molecular weight of the guar gum sample may be from hundreds of thousands to millions of Daltons. With the smaller molecular weights of the PHGG sample, the viscosity of the PHGG sample may be reduced and the solubility of the PHGG sample may be increased, than that of the guar gum sample. In some embodiments, the viscosity of the guar gum sample may be about 250 times greater than PHGG. In some embodiments, the PHGG sample may keep the biological regulatory activity that the regular guar gum sample has and the plurality of polysaccharides in the PHGG sample may have additional biological activities that the regular guar gum sample does not have. For example, the PHGG sample may promote bowel regularity and selectively improve the health of beneficial gut bacteria. As another example, the PHGG sample may promote healthy blood sugar and cholesterol levels and provide a satiety effect by regulating hunger/fullness hormones such as cholecystokinin. Accordingly, the PHGG sample may reach a balance between the viscosity and the bioregulatory activity thereof, such that the PHGG sample can be added in cereals, juices, shakes, yogurt, meal replacements, soups, baked goods, dietary supplements, and medical foods and/or used as a fiber source in enteral nutrition products, thereby solving the limitation of the usage of the regular guar gum sample that caused by the high viscosity.

### EXAMPLES

### Example 1: Enzymatic Hydrolysis of a Guar Gum Sample

A gene (Uniprot #G1K3N4) encoding a glycosyl hydrolase 5 (cellulase A) family beta-mannosidase (i.e., β-mannanase) (SagMan) from Salipaludibacillus agaradhaerens was synthesized. An enzyme (i.e., SagMan) was produced in Bacillus subtilis cells using previously described methods (e.g., a method disclosed in Ji M et al., Enzyme Microb Technol. 2021 March; 144:109726). A protein of SagMan was secreted into an extracellular medium and filtered culture medium (also referred to as SagMan culture medium) was used for the enzymatic hydrolysis of a guar gum sample (Sigma G4129).

The guar gum sample was subjected to enzymatic hydrolysis using SagMan at a selected concentration of 1 mL filtered culture medium per 100 g guar gum. The enzymatic hydrolysis was performed at a pH of 6.0 and a temperature of 50° C. for the time of 2 hours. Firstly, the SagMan culture medium (i.e., the filtered culture medium) was added based on the selected concentration to 25 mM phosphate buffer at pH 6.0 to obtain a mannanase mixture. After the addition of the SagMan culture medium, the guar gum sample in powder was sprinkled in a vortex of the mannanase mixture using a laboratory stirrer at 1000 rpm. The enzymatic hydrolysis of the guar gum sample was then carried out using a shaking incubator at 50° C. and 1000 rpm for 2 hours to obtain a reaction mixture. The viscosity of a suspension of the reacted guar gum (i.e., the reaction mixture) dropped to about 20 mPa·s to 120 mPa·s at a shear rate of about 700 per second. The suspension was heated to about 95° C. for 10 minutes to inactivate the enzyme (i.e., SagMan), and then centrifuged at 16000 g to remove insoluble substance(s) in the reaction mixture, for obtaining a PHGG sample with the viscosity at about 10 mPa·s to 40 mPa·s at a shear rate of about 700 per second (also referred to as a first PHGG sample).

Similar to the first PHGG sample, a second PHGG sample was obtained by performing enzymatic hydrolysis on a guar gum sample using PpoMan; a third PHGG sample was obtained by performing enzymatic hydrolysis on a guar gum sample using PleMan; a fourth PHGG sample was obtained by performing enzymatic hydrolysis on a guar gum sample using XccMan; and a fifth PHGG sample was obtained by performing enzymatic hydrolysis on a guar gum sample using RspMan.

### Example 2: Molecular Weight Analysis of the PHGG Samples in Example 1

After the enzymatic hydrolysis of the guar gum samples as described in EXAMPLE 1, a supernatant of the suspension containing soluble carbohydrates (i.e., each of the first PHGG sample, the second PHGG sample, the third PHGG sample, the fourth PHGG sample, and the fifth PHGG sample) was conducted for molecular weight analysis using Agilent 1200 HPLC equipped with Tosoh Bioscience TSKgel G4000PWXL column (eluant: H2O; flow rate: 1.0 mL/min; temperature: 25° C.; detection: RI). Dextran standards (Mw 3,050 Da, 12.6 kDa, 63.3 kDa, and 102 kDa) were ordered from the National Institute of Metrology, China, and were analyzed by HPLC under the same condition. Data of log (Mw, dextran standards) vs. retention time (Rt) was plotted to obtain a straight line and a linear regression equation was determined. The average molecular weight of the hydrolyzed guar gum (i.e., each of the first PHGG sample, the second PHGG sample, the third PHGG sample, the fourth PHGG sample, and the fifth PHGG sample) was determined from the linear regression equation based on an analytical curve determined by the HPLC. The percentage of each of a plurality of polysaccharides in each of the PHGG samples was calculated based on the area under the analytical curve.

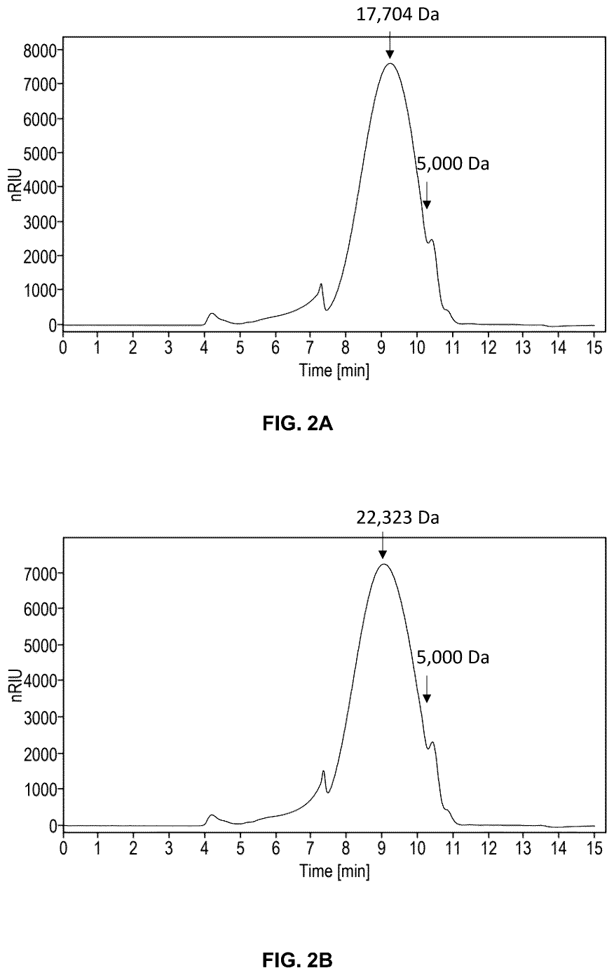

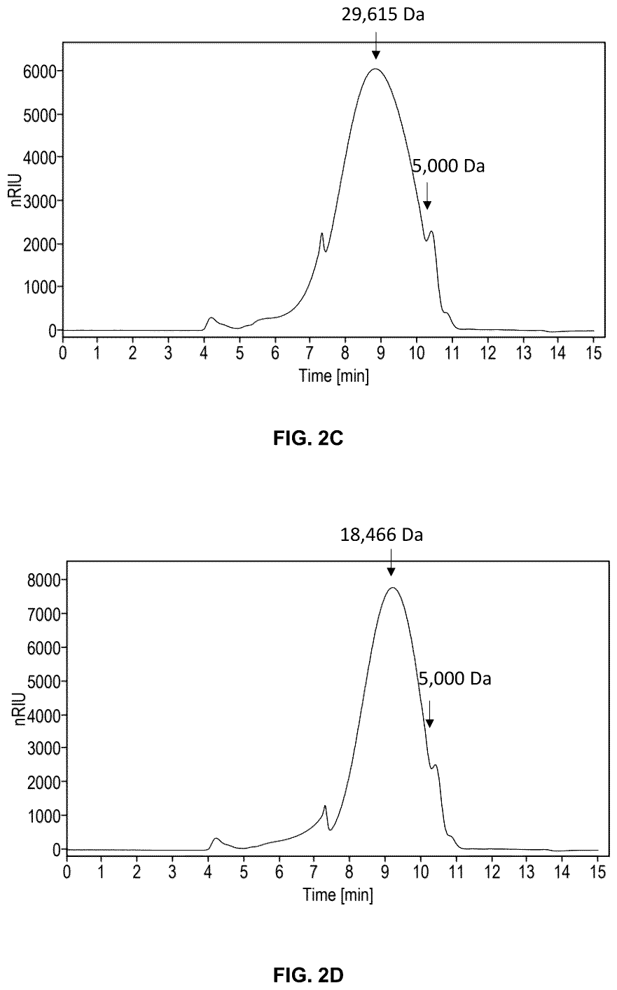

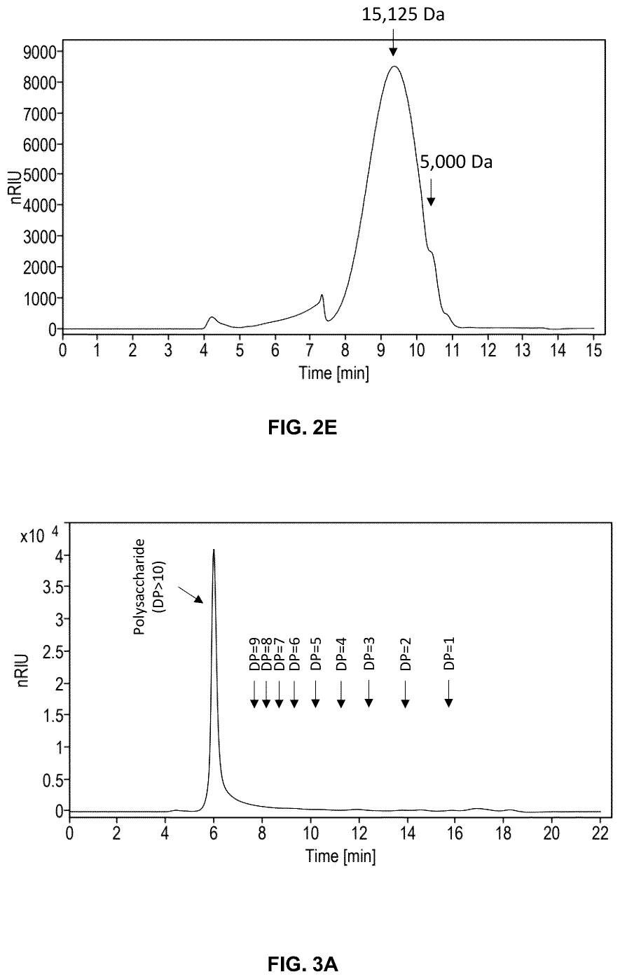

FIGS. 2A-2E illustrate the analytical curves determined by the HPLC in EXAMPLE 2. As shown in FIG. 2A, the average molecular weight of the first PHGG sample obtained from EXAMPLE 1 was about 17,704 Da, and more than 80% (i.e., >80%) by weight of the polysaccharides had molecular weights higher than 5,000 Da. As shown in FIG. 2B, the average molecular weight of the second PHGG sample obtained from EXAMPLE 1 was about 22,323 Da, and more than 85% (i.e., >85%) by weight of the polysaccharides had molecular weights higher than 5,000 Da. As shown in FIG. 2C, the average molecular weight of the third PHGG sample obtained from EXAMPLE 1 was about 29,615 Da, and more than 85% (i.e., >85%) by weight of the polysaccharides had molecular weights higher than 5,000 Da. As shown in FIG. 2D, the average molecular weight of the fourth PHGG sample obtained from EXAMPLE 1 was about 18,466 Da, and more than 80% (i.e., >80%) by weight of the polysaccharides had molecular weights higher than 5,000 Da. As shown in FIG. 2D, the average molecular weight of the fifth PHGG sample obtained from EXAMPLE 1 was about 15,125 Da, and more than 75% (i.e., >75%) by weight of the polysaccharides had molecular weights higher than 5,000 Da.

### Example 3: Oligosaccharide Analysis (i.e., Low Molecular Weight Sugar Analysis) of the PHGG Samples in Example 1

After the enzymatic hydrolysis of the guar gum samples as shown in EXAMPLE 1, the supernatant of the suspension containing soluble carbohydrates (i.e., each of the first PHGG sample, the second PHGG sample, the third PHGG sample, the fourth PHGG sample, and the fifth PHGG sample) was conducted for high-resolution oligosaccharide analysis using Agilent 1200 HPLC equipped with Bio-Rad Aminex HPX-42A column (eluant: H2O; flow rate: 0.6 mL/min; temperature: 85° C.; detection: RI).

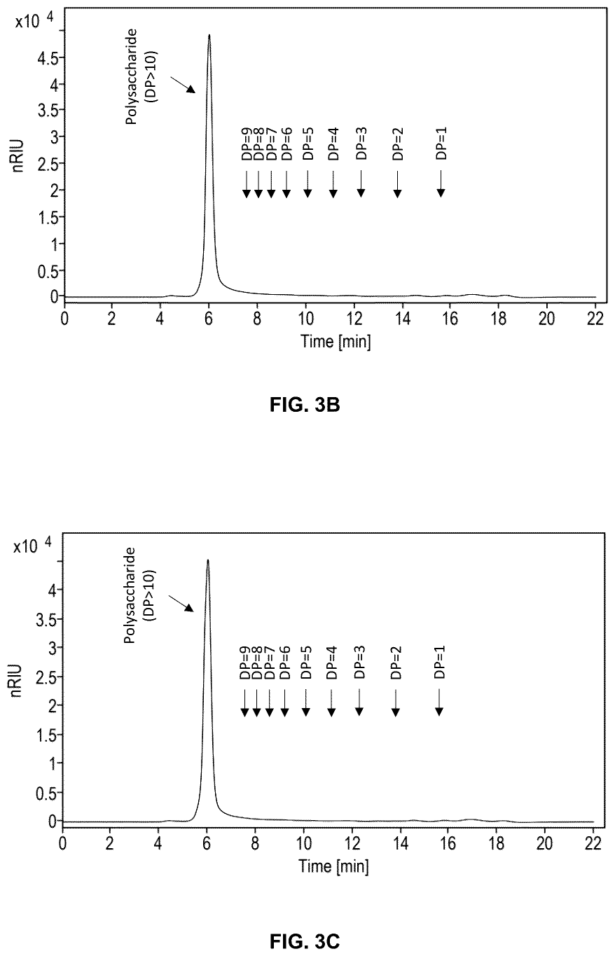

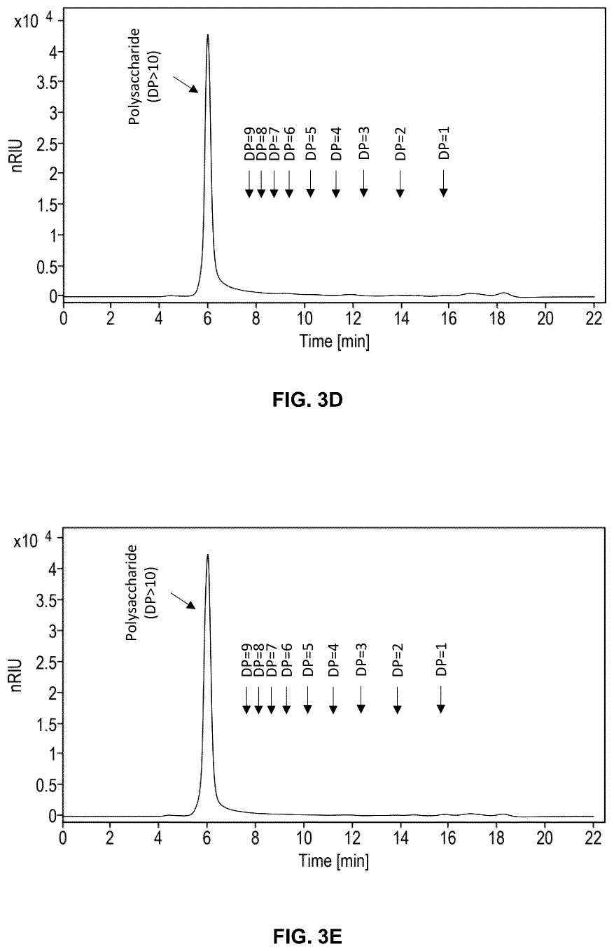

FIGS. 3A-SE were analytical curves determined by the HPLC in EXAMPLE 3. The analytical curves in FIG. 3A-3E illustrated results of soluble carbohydrate analysis of the first PHGG sample, the second PHGG sample, the third PHGG sample, the fourth PHGG sample, and the fifth PHGG sample obtained from EXAMPLE 1. As shown in FIG. 3A, the PHGG sample mainly included a substantial amount of soluble polysaccharides with DP>10, and less than 3% of oligosaccharides with DP<10 were generated. As shown in FIG. 3B, the second PHGG sample mainly included a substantial amount of soluble polysaccharides with DP>10, and less than 3% of oligosaccharides with DP<10 were generated. As shown in FIG. 3C, the third PHGG sample mainly included a substantial amount of soluble polysaccharides with DP>10, and less than 3% of oligosaccharides with DP<10 were generated. As shown in FIG. 3D, the fourth PHGG sample mainly included a substantial amount of soluble polysaccharides with DP>10, and less than 3% of oligosaccharides with DP<10 were generated. As shown in FIG. 3E, the fifth PHGG sample mainly included a substantial amount of soluble polysaccharides with DP>10, and less than 3% of oligosaccharides with DP<10 were generated.

### Example 4: Enzymatic Hydrolysis of a Guar Gum Sample Under Different Reaction Time

A mannanase mixture containing SagMan was prepared using 25 mM phosphate buffer at pH 6.0. A guar gum sample in powder was sprinkled in the vortex of the mannanase mixture using the laboratory stirrer at 1000 rpm. Enzymatic hydrolysis of the guar gum sample was carried out at 50° C. and 1000 rpm using the shaking incubator for 0.5, 1, 1.5, 2, 6, and 24 hours to obtain reaction mixtures. The suspension of each reaction mixture was heated to about 95° C. for 10 minutes to inactivate SagMan in each reaction mixture, and then centrifuged at 16000 g to remove insoluble substance(s) in each reaction mixture, for obtaining a PHGG sample corresponding to each reaction time.

Part of the supernatant containing soluble carbohydrates was conducted for SEC analysis using Agilent 1200 HPLC equipped with Tosoh Bioscience TSKgel G4000PW_{XL} column (Eluant: H₂O; Flow rate: 1.0 mL/min; Temperature: 25° C.; Detection: RI), which is similar to that described in EXAMPLE 2.

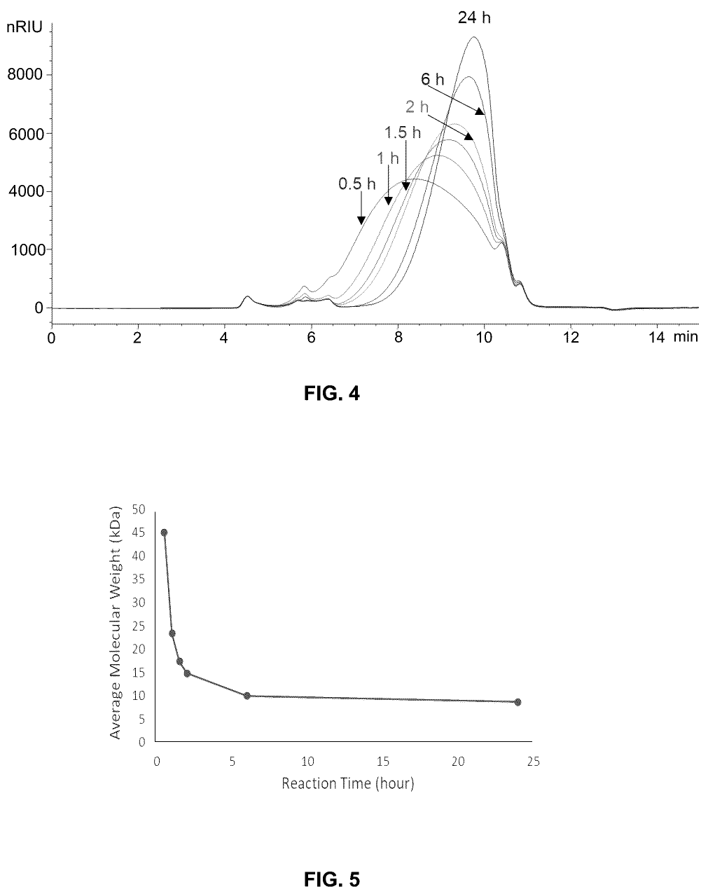

FIG. 4 shows the SEC analysis of the PHGG samples. FIG. 5 shows the determined average molecular weights of the PHGG samples. The average molecular weights of the PHGG samples obtained under different reaction time were determined from the linear regression equation (as described in EXAMPLE 2) based on the curves illustrated in FIG. 4 and used to determine a curve in FIG. 5. As shown in FIG. 5, the average molecular weight of the PHGG sample reduced quickly at the first 2 hours. The average molecular weight of the PHGG sample wouldn't change much after reacting for 6 hours or longer. The final average molecular weight was around 15 kDa-18 kDa after the 2-hour reaction. The final average molecular weight was around 10 kDa after the 24-hour reaction. Accordingly, though the reaction time extends, the final average molecular weight of the PHGG sample may be kept at a desired average molecular weight (i.e., greater than 5,000 Da) at which the PHGG sample may reach a good balance between the viscosity and the bioregulatory activity of the PHGG sample.

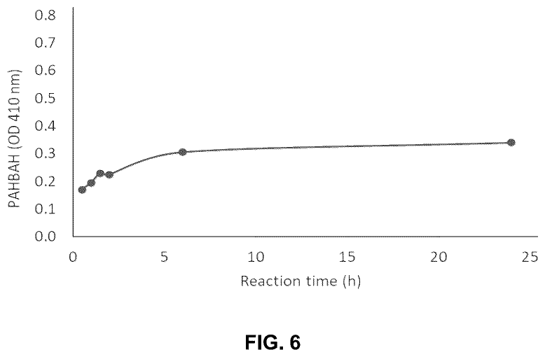

The other part of the supernatant was conducted for reducing sugar analysis using a previously described PAHBAH method (e.g., the PAHBAH method disclosed in Lever, M. (1972). Analytical Biochemistry 47:248). FIG. 6 shows the result of the reducing sugar analysis. As shown in FIG. 6, the reducing sugar slightly increased as the reaction time increased, which also indicates that very few oligosaccharides were generated by the enzymatic hydrolysis using SagMan.

The present disclosure has been described in detail above in conjunction with the accompanying drawings and embodiments. However, those skilled in the art can understand that changes, modifications, substitutions, combinations, and simplifications made without departing from the concept of the present disclosure should be equivalent alternatives, thereby forming multiple specific embodiments, that are within the scope of common variations of the present disclosure.

What is claimed is:

1. A method for preparing a partially hydrolyzed guar gum (PHGG) sample, comprising:
performing enzymatic hydrolysis on a guar gum sample using a β-mannanase to obtain a reaction mixture; and
processing the reaction mixture to obtain the PHGG sample, wherein the PHGG sample includes a plurality of polysaccharides, and more than 70% by weight of the plurality of polysaccharides have molecular weights higher than 5,000 Daltons, the PHGG sample includes one or more oligosaccharides that have degrees of polymerization (DP) less than or equal to 10, and a mass concentration of all the one or more oligosaccharides in the PHGG sample is less than 3%.

2. The method of claim 1, wherein the β-mannanase includes a glycosyl hydrolase 5 family β-mannanase that is absent of carbohydrate-binding modules (CBMs).

3. The method of claim 2, wherein the β-mannanase includes at least one of SagMan derived from Salipaludibacillus agaradhaerens, PpoMan derived from Paenibacillus polymyxa, PleMan derived from Paenibacillus lentus, XccMan derived from Xanthomonas campestris pv. campestris, or RspMan derived from Ruminiclostridium sp., the SagMan having an amino acid sequence of SEQ ID NO: 1, the PpoMan having an amino acid sequence of SEQ ID NO: 2, PleMan having an amino acid sequence of SEQ ID NO: 3, XccMan having an amino acid sequence of SEQ ID NO: 4, and RspMan having an amino acid sequence of SEQ ID NO: 5.

4. The method of claim 3, wherein the β-mannanase includes the SagMan derived from the Salipaludibacillus agaradhaerens.

5. The method of claim 1, wherein more than 80% by weight of the plurality of polysaccharides have molecular weights higher than 5,000 Daltons.

6. The method of claim 5, wherein more than 85% by weight of the plurality of polysaccharides have molecular weights higher than 5,000 Daltons.

7. The method of claim 1, wherein the one or more oligosaccharides have molecular weights less than 1,800 Daltons.

8. The method of claim 1, wherein the performing the enzymatic hydrolysis on the guar gum sample using the β-mannanase to obtain the reaction mixture includes:
preparing a mannanase mixture including the β-mannanase and having a preset PH;
adding, by stirring the mannanase mixture under a preset temperature, the guar gum sample into the mannanase mixture to obtain a gel mixture, the guar gum sample being in powder; and
reacting the gel mixture for a preset time to obtain the reaction mixture.

9. The method of claim 8, wherein a mass concentration of the guar gum sample in the gel mixture is from 5% to 20%.

10. The method of claim 9, wherein the mass concentration of the guar gum sample in the gel mixture is from 8% to 20%.

11. The method of claim 10, wherein the mass concentration of the guar gum sample in the gel mixture is from 10% to 15%.

12. The method of claim 8, wherein the preset time is from 1 hour to 24 hours.

13. The method of claim 12, wherein the preset time is from 1 hour to 4 hours.

14. The method of claim 1, wherein the PHGG sample has an average molecular weight from 10 kDa to 30 kDa, and in the PHGG sample, a mass concentration of the one or more oligosaccharides having DP less than or equal to 10 is less than 3%.

15. The method of claim 14, wherein the PHGG sample has the average molecular weight from 15 kDa to 25 kDa.

16. The method of claim 1, wherein the processing the reaction mixture includes:
inactivating the β-mannanase in the reaction mixture.

17. The method of claim 16, wherein the processing the reaction mixture further includes:
performing at least one of a centrifugal operation, a filtering operation, a sterilization operation, a concentration operation, or a dry operation on the reaction mixture.

18. The method of claim 1, further comprising:
analyzing the PHGG sample by at least one of size exclusion chromatography (SEC), high performance liquid chromatography (HPLC), a reducing sugar analyzation, or ion chromatography (IC).

19. A composition containing a partially hydrolyzed guar gum (PHGG) sample, wherein the PHGG sample is prepared by performing enzymatic hydrolysis on a guar gum sample using a β-mannanase to obtain a reaction mixture and processing the reaction mixture, wherein the PHGG sample includes a plurality of polysaccharides, and more than 70% by weight of the plurality of polysaccharides have molecular weights higher than 5,000 Daltons, the PHGG sample includes one or more oligosaccharides that have degrees of polymerization (DP) less than or equal to 10, and a mass concentration of all the one or more oligosaccharides in the PHGG sample is less than 3%.

20. The composition of claim 19, wherein the β-mannanase includes a glycosyl hydrolase 5 family β-mannanase that is absent of carbohydrate-binding modules (CBMs).
